(19) United States
(12) Patent Application Publication
(10) Pub. No.: US 2026/0325005 A1
(43) Pub. Date: Oct. 22, 2026

(54) METHOD AND SYSTEM FOR ASSESSING LUNG CHARACTERISTICS USING ELECTRICAL IMPEDANCE TOMOGRAPHY IN HIGH FREQUENCY VENTILATION

(71) Applicant: Timpel Medical B.V., Eindhoven (NL)

(72) Inventors: Daniela Davi Madureira Iope, São Paulo (BR); Alexandre Bezerra de Melo, São Paulo (BR)

(21) Appl. No.: 19/655,258

(22) Filed: Apr. 22, 2026

Related U.S. Application Data
(60) Provisional application No. 63/792,557, filed on Apr. 22, 2025.

(51) Int. Cl. A61B 5/085; A61B 5/00; A61M 16/00

(52) U.S. Cl. CPC A61B 5/085; A61B 5/743; A61M 16/024

(57) ABSTRACT

A method for assessing regional lung characteristics of a patient undergoing high-frequency ventilation using an impedance-based device is disclosed. The method includes determining a first impedance quantification from first electrical impedance data associated with at least one region of interest and acquired prior to a first ventilatory maneuver, determining a second impedance quantification from second electrical impedance data associated with the at least one region of interest and acquired following the first ventilatory maneuver, obtaining at least one parameter associated with the first ventilatory maneuver, determining a first regional lung characteristic associated with the at least one region of interest based at least in part on the first impedance quantification, the second impedance quantification, and the parameter, and causing a change in configuration of a high-frequency mechanical ventilator. Related methods and systems are also disclosed.

### CROSS-REFERENCE TO RELATED APPLICATIONS

This application claims the benefit under 35 U.S.C. § 119 (e) of U.S. Provisional Patent Application Ser. No. 63/792,557, filed Apr. 22, 2025, the disclosure of which is hereby incorporated herein in its entirety by this reference.

### TECHNICAL FIELD

This disclosure relates generally to electrical impedance tomography in high frequency ventilation.

### BACKGROUND

Electrical Impedance Tomography (EIT) is a non-invasive imaging method that may be used to generate images or physiological measurements associated with a section of a subject (e.g., a patient or object) by collecting data using electrodes disposed along the perimeter of the subject. Potential applications of EIT include medical applications in which real-time tomographic images or physiological measurements of the human body may be useful. As an example, EIT may be used to perform non-invasive monitoring of cardio-respiratory systems, which may be particularly useful in patients under treatment in intensive care unit (ICU) environments, including patients undergoing mechanical ventilation. In some ventilatory contexts, such as high-frequency ventilation (HFV), small tidal volumes and rapid oscillatory pressure changes may complicate assessment of lung mechanics using conventional airway-based measurements.

In an EIT system, electrical signals (e.g., electric currents) may be injected into a perimeter of the subject being imaged (e.g., a patient torso). Electrical characteristics (e.g., voltages, electric potentials) resulting from the injected electrical signals may be collected at the perimeter of the subject. From the collected data, maps with an estimate of the electrical impedance in each portion (e.g., pixel, voxel) of the subject may be generated or reconstructed. This process may be repeated over time, and the sequence of tomographic images may form a “video.” The video may be employed to provide physiological information to a physician or other clinical practitioner. Processing and/or quantification of the EIT data may also be performed to calculate physiological parameters and/or figures of merit, which may assist assessment of ventilation distribution and regional lung characteristics, including in situations in which conventional flow- and pressure-based measurements may be difficult to interpret.

### BRIEF DESCRIPTION OF THE DRAWINGS

For a detailed understanding of the disclosure, reference should be made to the following detailed description, taken in conjunction with the accompanying drawings, in which like elements have generally been designated with like numerals, and wherein:

FIG. 1 illustrates a schematic diagram for a method to perform high frequency ventilation assessments of a patient in accordance with embodiments of the disclosure;

FIG. 2 illustrates a schematic diagram of an EIT system in accordance with embodiments of the disclosure;

FIG. 3 illustrates a schematic diagram for the performance of impedance measurements in accordance with embodiments of the disclosure;

FIG. 4 illustrates a plot of example impedance quantifications and ventilatory maneuvers that may be used to assess at least one of regional lung compliance or regional lung resistance in accordance with embodiments of the disclosure;

FIG. 5 is a simplified schematic model representative of the system in accordance with embodiments of the disclosure;

FIG. 6A represents a plethysmogram in accordance with embodiments of the disclosure;

FIG. 6B shows the separation of HFV and spontaneous components of the plethysmogram of FIG. 6A in accordance with embodiments of the disclosure;

FIG. 6C shows HFV components of the plethysmogram of FIG. 6A in accordance with embodiments of the disclosure;

FIG. 7 is a flow diagram of a process for obtaining regional impedance data representing HFV ventilation in accordance with embodiments of the disclosure;

FIG. 8 is a block diagram of impedance quantifications performed in an embodiment in accordance with embodiments of the disclosure;

FIG. 9A illustrates an embodiment of a graphical user interface in accordance with embodiments of the disclosure;

FIG. 9B illustrates an embodiment of a graphical user interface in accordance with embodiments of the disclosure; and

FIGS. 10A and 10B illustrate user interface elements of an EIT device in accordance with embodiments of the disclosure.

### BRIEF SUMMARY

Embodiments of the disclosure include, a method for assessing regional lung characteristics of a patient undergoing high-frequency ventilation using an impedance-based device. The method includes determining a first impedance quantification from first electrical impedance data associated with at least one region of interest and acquired prior to a first ventilatory maneuver, determining a second impedance quantification from second electrical impedance data associated with the at least one region of interest and acquired following the first ventilatory maneuver, obtaining at least one parameter associated with the first ventilatory maneuver, determining a first regional lung characteristic associated with the at least one region of interest based at least in part on the first impedance quantification, the second impedance quantification, and the at least one parameter associated with the first ventilatory maneuver, and causing a change in a configuration of a high-frequency mechanical ventilator based at least in part on the first regional lung characteristic. The change in the configuration includes changing at least one of a frequency of oscillation, an inspiratory time, an inspiratory-to-expiratory relationship, an amplitude of oscillation, a mean airway pressure, or any combination thereof.

Another embodiment of the disclosure includes, a system for displaying slow-motion electrical impedance data associated with at least one region of interest of a patient undergoing high-frequency ventilation. The system includes an impedance-based device including a plurality of electrodes and a controller coupled to the plurality of electrodes. The controller includes an interface module configured to collect electrical impedance data from the plurality of electrodes, at least one processor, and at least one non-transitory computer-readable storage medium storing instructions thereon that, when executed by the at least one processor, cause the controller to acquire electrical impedance data corresponding to a plurality of oscillations, estimate, from the electrical impedance data, at least one of an amplitude or a phase associated with the at least one region of interest, generate a slow-motion image associated with the at least one region of interest by synthesizing a signal based at least in part on the at least one of the amplitude or the phase, and output, via at least one display of the impedance-based device, the slow-motion image.

Another embodiment of the disclosure includes, a method for simulating a regional lung response of a patient undergoing high-frequency ventilation using an impedance-based device. The method includes obtaining at least one of a first regional lung characteristic, a second regional lung characteristic, or a ventilatory figure of merit associated with at least one region of interest, calculating, based at least in part on the at least one of the first regional lung characteristic, the second regional lung characteristic, and the ventilatory figure of merit, a predicted regional lung response of the patient to a candidate change in a configuration of a high-frequency mechanical ventilator before the candidate change is applied, wherein the candidate change in the configuration of the high-frequency mechanical ventilator includes a change to at least one of a frequency of oscillation, an inspiratory time, an inspiratory-to-expiratory relationship, an amplitude of oscillation, a mean airway pressure, or any combination thereof, and outputting, via a display of the impedance-based device, the predicted regional lung response associated with the at least one region of interest.

Other embodiments of the disclosure include, a system for assessing regional lung characteristics of a patient undergoing high-frequency ventilation using an impedance-based device. The system includes an impedance-based device including a plurality of electrodes, and a controller coupled to the plurality of electrodes. The controller includes an interface module configured to collect electrical impedance data from the plurality of electrodes, at least one processor, and at least one non-transitory computer-readable storage medium storing instructions thereon that, when executed by the at least one processor, cause the controller to determine a first impedance quantification from first electrical impedance data associated with at least one region of interest and acquired prior to a first ventilatory maneuver, determine a second impedance quantification from second electrical impedance data associated with the at least one region of interest and acquired following the first ventilatory maneuver, obtain at least one parameter associated with the first ventilatory maneuver, and determine a first regional lung characteristic associated with the at least one region of interest based at least in part on the first impedance quantification, the second impedance quantification, and the at least one parameter associated with the first ventilatory maneuver, the first regional lung characteristic including one or more of regional lung compliance, regional lung resistance, or lung homogeneity. Determining the first impedance quantification includes determining one or more of a baseline of a plethysmogram, an offset of the plethysmogram, an average of the plethysmogram, a magnitude of oscillation, a phase of oscillation, and an asynchronous filling. Determining the first impedance quantification also includes correcting a phase of electrical impedance data corresponding to the first impedance quantification by applying at least one of a fractional sample delay algorithm, a phase shift in a frequency domain, finite impulse response filtering, or interpolation to the corresponding electrical impedance data.

### DETAILED DESCRIPTION

The illustrations presented herein are not actual views of any system for assessing lung characteristics using electrical impedance tomography in high frequency ventilation, or any component thereof, but are merely idealized representations, which are employed to describe embodiments of the invention.

As used herein, the singular forms following “a,” “an,” and “the” are intended to include the plural forms as well, unless the context clearly indicates otherwise.

As used herein, the term “may” with respect to a material, structure, feature, or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, any relational term, such as “first,” “second,” “top,” “bottom,” “upper,” “lower,” “above,” “beneath,” “side,” “upward,” “downward,” etc., is used for clarity and convenience in understanding the disclosure and accompanying drawings, and does not connote or depend on any specific preference or order, except where the context clearly indicates otherwise. For example, these terms may refer to an orientation of elements of any system for assessing lung characteristics using electrical impedance tomography in high frequency ventilation when utilized in a conventional manner. Furthermore, these terms may refer to an orientation of elements of any system for assessing lung characteristics using electrical impedance tomography in high frequency ventilation as illustrated in the drawings.

As used herein, the term “substantially” in reference to a given parameter, property, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or at least 99.9% met, or even 100.0 percent met.

As used herein, the term “about” used in reference to a given parameter is inclusive of the stated value and has the meaning dictated by the context (e.g., it includes the degree of error associated with measurement of the given parameter, as well as variations resulting from manufacturing tolerances, etc.). For example, “about” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

As used herein, “high-frequency ventilation” or “HFV” refers to mechanical ventilation delivered at frequencies above conventional respiratory frequencies, including, in some embodiments, high-frequency oscillatory ventilation (HFOV). For example, HFV or HFOV may be delivered at frequencies greater than about 5 Hz, such as in a range from about 5 Hz to about 20 Hz, although other frequencies above conventional respiratory frequencies may also be used.

As used herein, “regional lung characteristic” may include regional lung compliance, regional lung resistance, ventilation-related impedance change (AZ), plethysmogram baseline, plethysmogram offset, plethysmogram average, amplitudes of oscillation, asynchronous filling, phase or delay, homogeneity of regional ventilation, homogeneity of regional oscillation distribution, lung homogeneity, or combinations thereof.

As used herein, “regional lung homogeneity” or “lung homogeneity” refers to how evenly oscillations or ventilation-related changes in impedance are distributed across lung regions. Homogeneous lungs may exhibit similar tidal changes or magnitudes of oscillation changes across regions, or similar compliances or resistances across regions.

As used herein, ‘regional asynchronous filling’ or ‘asynchronous filling’ refers to a temporal inhomogeneity in which different lung regions change volume or respond to an oscillation caused by a high-frequency ventilator at different times or with different timing patterns during a cycle. Regional asynchronous filling may be assessed, for example, as time-to-peak or time-to-50% (e.g., a time from the start of inspiration to a pixel's maximal impedance change), delay (e.g., when a pixel's impedance begins to rise with a delay relative to a reference), phase angle or regional phase shift (e.g., derived from cross-correlation or Fourier analysis between a pixel signal and a global signal), breath-by-breath standard deviation or coefficient of variation of timing metrics across pixels, pendelluft (e.g., regional impedance changes that occur without corresponding global volume change or with opposite-phase changes between adjacent regions), or a combination thereof.

References to “one embodiment” or “an embodiment” of the present disclosure are not intended to be interpreted as excluding the existence of additional embodiments that also incorporate the recited features. Furthermore, the phrase A “based on” B is intended to mean that A is at least partially based on B.

As discussed herein, references may be made to “electrical impedance data.” As described herein, electrical impedance data relates to data obtained from an electrical impedance tomography (EIT) system or an electrical impedance measurement device, or any other data that relates to electrical characteristics (e.g., impedance, resistivity, capacitance, inductance, permittivity, etc.) of the measured subject (e.g., a patient, an object). Electrical impedance data refers to raw (i.e., unprocessed) data, filtered data, and/or tomographically reconstructed data. Tomographic reconstructions from electrical impedance data may be specifically referred herein as “electrical impedance images” or “electrical impedance videos.”

Electrical impedance images may be further described as “absolute impedance images,” “differential impedance images,” and “volumetric images” of a subject. Absolute impedance images, as discussed herein, relates to images in which the representation (e.g., value) of each pixel or voxel is associated to the impedance of the corresponding portion of the subject being imaged. Differential impedance images, as discussed herein, relates to images in which the representation (e.g., value) of each pixel or voxel is associated to a change of the impedance of the corresponding portion of the subject relative to a baseline image. Volumetric images, as discussed herein, relates to images in which the representation (e.g., value) of each pixel or voxel is associated with a volume of a biological fluid (e.g., air, blood, water, tissue type). Moreover, it should be understood that the impedance images or videos, as referred herein, may be tomographic images that may be two-dimensional (2-D) or three-dimensional (3-D), as well as tomographic movies with a time dimension.

As discussed above, due to the nature of the measurements and the speed of data acquisition and processing, electrical impedance-based systems, such as EIT, can be used to generate high-frequency, real-time physiological data that may be of assistance to a physician during certain diagnostic and/or clinical procedures. Moreover, since impedance-based systems are generally non-invasive, they can be used with a wider range of patient populations in a broad variety of conditions.

According to embodiments described herein, EIT may be used to assess regional lung characteristics in patients undergoing HFV. The disclosed approaches may facilitate evaluation of how changes in one or more ventilatory parameters affect regional impedance behavior, regional distribution of ventilation, regional timing, and regional mechanical properties. This information may facilitate configuration or adjustment of HFV parameters, including frequency, MAP, oscillatory amplitude (AP), inspiratory time, inspiratory-to-expiratory ratio, or combinations thereof.

In conventional mechanical ventilation, dynamic lung compliance Cdyn may be expressed as:

Cdyn=AV/AP (1) where AV represents a change in tidal volume and AP represents a pressure difference associated with ventilation.

In EIT-based regional assessment, regional compliance Cregional may be expressed as:
Cregional=AZ/AP (2) where AZ represents a regional change in impedance used as a surrogate for regional volume change. However, during HFV, direct application of airway-based pressure and flow measurements may be complicated by the oscillatory nature of the signal, small tidal volumes, circuit effects, and chest-wall interactions. The methods and systems disclosed herein estimate regional lung compliance, regional lung resistance, or both, based on regional impedance quantifications before and after a ventilatory maneuver, together with one or more parameters associated with that maneuver.

EIT may provide information from multiple perspectives relevant to HFV. In some embodiments, EIT is used to assess distribution of the magnitude and/or phase of oscillations to determine whether lung regions are being ventilated more or less homogeneously. In some embodiments, EIT is used to assess distribution of tidal volume associated with spontaneous breaths or conventional ventilation that coexist with HFV. In some embodiments, EIT is used to assess a combined effect of HFV and spontaneous or conventional ventilation, to identify silent regions to one or more ventilation modes, to identify regions exhibiting delayed inflation or deflation, to assess regional lung compliance and regional lung resistance, and to simulate how selected ventilatory maneuvers may affect regional volume distribution and timing.

In some embodiments, the EIT system is connected to a high-frequency ventilator and receives information regarding ventilator configuration. In some embodiments, the EIT system instead or additionally detects one or more ventilator-configuration parameters by analyzing the impedance data itself. In some embodiments, once regional lung characteristics are estimated, adjustments are made to MAP, frequency, oscillatory amplitude, inspiratory time, inspiratory-to-expiratory ratio, or combinations thereof, and the process may be repeated to reassess regional lung behavior at a new setting.

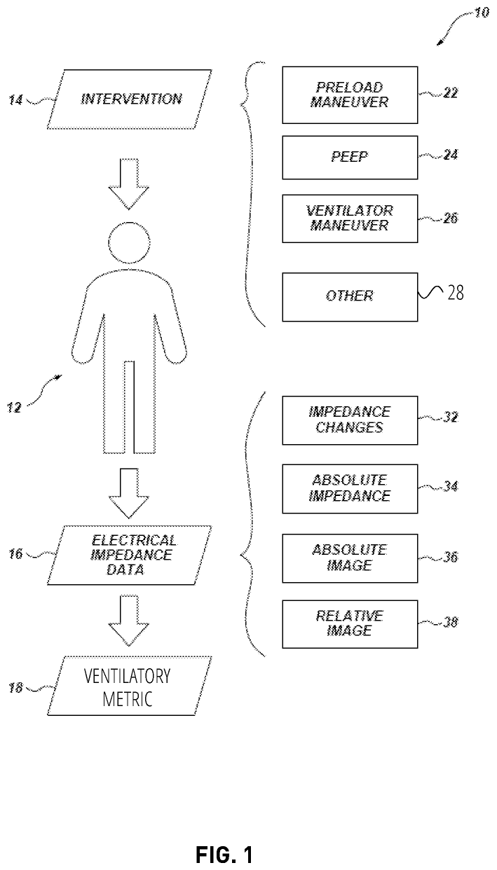

FIG. 1 illustrates a schematic diagram for a method 10 to perform high frequency ventilation assessments of a patient 12. In the method 10, the patient 12 may be subjected to an intervention 14 that may cause ventilation changes to the patient. The patient 12 may be monitored using an impedance-based device that collects electrical impedance data 16. The electrical impedance data 16 may include data from prior to the intervention 14, during the intervention 14, and/or after the intervention 14. The electrical impedance data 16 may be processed to obtain one or more ventilatory metrics 18. The metrics may be displayed to a practitioner or may be compared against threshold values to generate alarm signals or other types of display, as detailed below.

The intervention 14 may be any controlled intervention that can generate ventilatory changes in the patient 12. In certain situations, a change in frequency, MAP, amplitude, inspiratory time, or any combination of the foregoing may be used. In some embodiments, the impedance-based system is configured to provide instructions to the practitioner during the maneuver based on the figures of merit calculated in real time. The electrical impedance data 16 may be recorded in accordance with the provided instructions and may include metadata associated with the provided instructions.

In some embodiments, the impedance-based system is connected to the mechanical ventilator to receive respiratory data from the ventilator, which may be included as metadata for the electrical impedance data 16. In some embodiments, the impedance-based system is configured to control the mechanical ventilator to perform the assessment. The electrical impedance data 16 may be recorded during the assessment in accordance with a preload maneuver 22, a positive end-expiratory pressure (PEEP) maneuver 24, a ventilator maneuver 26 or other maneuver 28 (e.g., movement of the patient or a change in position of the patient), and may include respiratory data and/or ventilator controls as associated metadata.

The electrical impedance data 16 that can be used for the assessment of the HFV may include the relative impedances 32, absolute impedances 34, absolute tomographic images 36, relative tomographic images 38, and other such images. In some embodiments, the ventilatory metrics 18 are separately calculated in different regions of interest (ROIs) of the subject. In those embodiments, the tomographic images 36, 38 may be used, as detailed below.

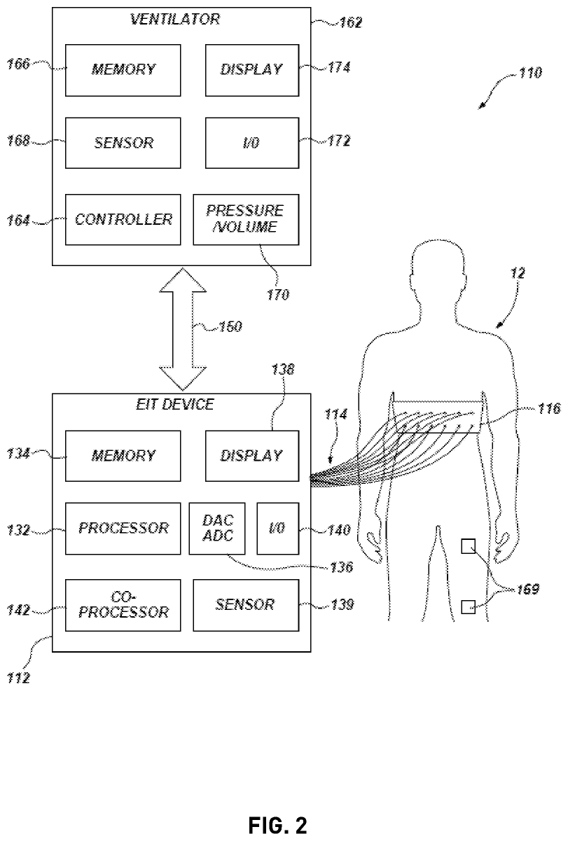

Regarding FIG. 2, the EIT device 112 may be coupled through electrical leads 114 to an electrode belt 116 that may be placed on the patient 12. In some embodiments, the EIT device 112 includes an acquisition module and the electrode belt 116, and may optionally be used with a pneumotachograph. The electrode belt 116 may have one or more rows of electrodes, disposed along the perimeter of the electrode belt 116. In some embodiments, the electrode belt 116 is a flexible belt including from about 16 to about 32 medical-grade electrodes. The electrodes of the electrode belt 116 may be spatially distributed along the perimeter of the torso of patient 12. In some embodiments, the electrode belt 116 is positioned on the chest of the patient 12 at the axillary line, with a center of the electrode belt 116 aligned with the xiphoid process. In a particular embodiment, impedance data may be acquired at about 50 frames per second while ventilation parameters are acquired at about 100 measurements per second. The EIT device 112 may inject electrical currents through electrodes of the electrode belt 116 and may collect the resulting electrical voltages. The EIT device may further include one or more sensors 139 for collecting data. From the collected data, the EIT device 112 may calculate one or more ventilatory metrics.

The EIT device 112 may perform filtering, reconstruction, and/or quantification algorithms, as detailed below. To that end, the computational device may include one or more processors 132 and one or more processing memory devices 134 (e.g., cache memory, random access memory (RAM)) to facilitate execution of the algorithms. The memory devices 134 may also include one or more protocols for performing the ventilatory assessment. The protocols may include instructions to be provided to a practitioner and instructions to create or associate metadata to the impedance data. The memory devices 134 may also include one or more segmentation algorithms to automatically determine ROIs used in the ventilatory assessment. In some embodiments, the segmentation algorithms include principal component analysis (PCA).

In some embodiments, some or all of the algorithms described herein are performed by a co-processor 142, which may be a second processor, a specialized processor such as a graphical processing unit (GPU), an application-specific integrated circuit (ASIC) designed to perform the algorithms, a field-programmable gate array (FPGA) containing configuration program that performs the algorithms, or any other dedicated electronic device.

The EIT device 112 may also include an interface module 136 to control the electrical currents injected into the electrical leads 114 and to measure the voltages between the electrical leads 114. The interface module 136 may include, among other things, analog signal generators, analog-to-digital converters, digital-to-analog converters, digital signal processors, filters, and impedance matching circuitry, to improve signal-to-noise ratio and decrease crosstalk.

In some embodiments, the EIT device 112 includes a display 138, which may be used to provide reconstructed images, charts, and/or indices, as well as diagnostic parameters that may be calculated from the EIT images. The display 138 may also be employed to provide instructions to a practitioner performing the assessment, as discussed above. In some embodiments, the display 138 includes or may be connected to a speaker or similar sound producing device to provide auditory alerts or auditory commands associated with the assessment.

In some embodiments, the EIT device 112 includes input/output interfaces 140 such as network interfaces, hard disk interfaces, and/or peripheral interfaces to send or receive data that may facilitate the operations of EIT, as discussed herein. For example, the EIT device 112 may be connected to the ventilator device 162 over an interface 150 that is coupled to the input/output interface 140. The interface 150 may be used to carry control commands and/or data that may be used to facilitate the assessment process, as discussed above.

In some embodiments, separation of a cardiac component and a respiratory component is ECG-gated, such that the filtering or extraction algorithm receives information associated with cardiac activity. By way of example, cardiac-activity information may be received from an electrocardiogram monitor, acquired through the EIT device, acquired using the same electrodes used for impedance measurements or different electrodes, determined by a posteriori synchronization, or obtained in any other suitable manner. In some embodiments, the EIT system communicates with an electrocardiogram monitor or other medical equipment to receive such information and to facilitate cardiac-component extraction, heartbeat synchronization, or both.

In certain configurations of the system 110, such as the one illustrated in FIG. 2, the patient 12 may be connected to the mechanical ventilator 162. The ventilator 162 may include a controller 164 (e.g., a processor, a microcontroller) that may, in conjunction with one or more memory devices 166, control the operations of the ventilator 162. In some embodiments, the memory devices 166 include protocols to perform ventilator maneuvers (e.g., ventilator maneuvers 26) to facilitate the assessment. The mechanical ventilator device 162 may include sensors 168, which may be a pressure sensor, flow sensor, and/or a carbon dioxide sensor. The ventilator device 162 may also include pumps 170, which may be pressure-controlled or volume-controlled pumps that provide respiratory support to the patient 12. The sensors 168 and pumps 170 may be controlled by the controller 164. In some embodiments, position sensors 169 are attached to the patient and facilitate measuring changes in the patient's position, which may simulate a change in load on the patient's lungs.

In some embodiments, the ventilator device 162 includes input/output interfaces 172 such as network interfaces, hard disk interfaces, and/or peripheral interfaces to send or receive data that may facilitate the respiratory support operations. The input/output interfaces 172 may be used to connect the ventilator 162 to the EIT device 112 over an interface 150, as discussed above. The interface 150 may be used to carry control commands or instructions from the EIT device 112, and to provide respiratory data from sensors 168 to the EIT device 112 to facilitate the assessment process, as discussed above.

In some embodiments, the ventilator device 162 includes a display 174, which may be used to provide respiratory charts, indices, and other physiological parameters associated with the respiratory support provided to the patient. In certain embodiments, the ventilator device 162 may be configured to facilitate assessments. In such devices, the display 174 may be configured to provide instructions to a practitioner performing the assessment, as discussed above. In some embodiments, the display 174 includes or may be connected to a speaker or similar sound-producing device to provide auditory alerts or auditory commands associated with the assessment.

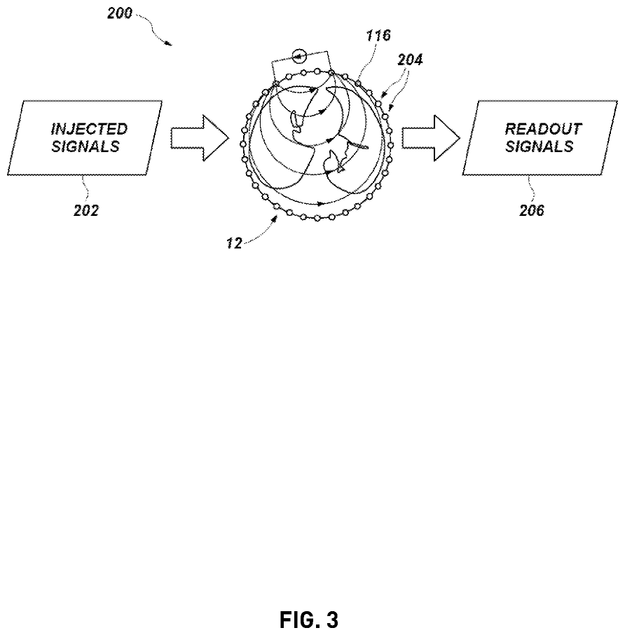

FIG. 3 illustrates a schematic diagram 200 for the performance of impedance measurements. Electrical signals 202 may be injected into pairs of electrodes 204 disposed along the electrode belt 116 around the patient 12. The electrical signals 202 may be injected in a pre-programmed order, and the resulting electrical potentials 206 between pairs of electrodes 204 may be read out. The collection of electrical potentials 206 may form the raw impedance data that may be used for assessment discussed herein.

The collected electrical potentials 206 may be used to perform tomographic reconstruction to obtain a series of impedance images. The tomographic reconstruction may employ an algorithm that provides absolute or relative impedance images. The reconstruction algorithm may include filters or other signal processing tools to mitigate noise and other artifacts. The reconstruction algorithm may be model-based algorithms. In some embodiments, the impedance images represent impedance change in an axial plane or slice defined by a position of the electrodes around the thorax of the patient 12.

The series of impedance images may be further filtered to separate individual component contributions. For example, a respiratory component and/or a cardiac component may be extracted from the impedance images, and high frequency component from HFV may be extracted from the respiratory component. Since the respiratory frequency is usually lower than the cardiac frequency, the filtering algorithm may employ frequency-based filtering over the time dimension of an impedance video. Examples of frequency-based filtering include high-pass filtering, low-pass filtering, band-pass filtering, Fourier-based filtering, moving average filters, autoregressive filters, autoregressive moving average (ARMA) filters, wavelet filters, state-space filters, or any other filter capable of separating components based on their frequency. Moreover, since perturbations from cardiac events and respiratory events are concentrated in different regions, the filtering algorithm may employ ROIs to facilitate the extraction of the cardiac component. It should be understood that any filtering algorithm capable of providing a quantifiable cardiac component can be used, or separating it from the respiratory component, and separating high frequency component from spontaneous or standard ventilation component, which may be dominant.

As discussed above, the impedance data may be accompanied by metadata obtained by, for example, positional sensors, video images, mechanical ventilators, and/or practitioner inputs. The metadata might facilitate the quantification of the physiological parameters. The metadata may provide, for example, time information (e.g., time of initiation of the maneuver, time of termination of the maneuver, duration of the maneuver), maneuver-specific information (e.g., type of maneuver), ventilator-specific information (e.g., frequency, MAP, oscillatory signal amplitude, respiratory flow, respiratory pressure, time of inspiration, time of expiration, spontaneous breath event), and/or other physiological information (e.g., pulse, oxygen concentration, heart beat markers). The metadata may be used to perform, for example, cardiac component extraction (e.g., heartbeat synchronization), automatic segmentation of ROIs, filtering and/or extraction of the cardiac component, and/or calculations of the figures of merit.

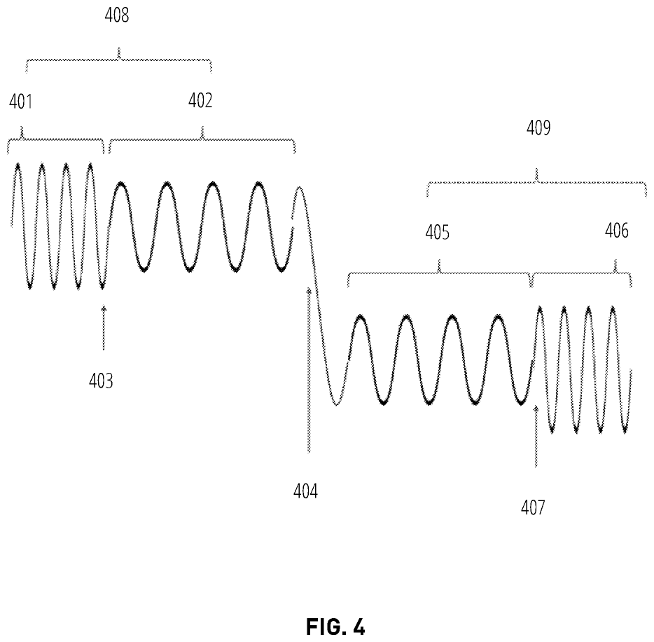

FIG. 4 illustrates a schematic oscillatory impedance signal from which impedance quantifications may be obtained to assess regional lung compliance, regional lung resistance, or both. While a patient undergoes HFV at a given MAP, for example, at a frequency of about 8 Hz and a MAP of about 10 cmH₂O, the EIT system may measure the frequency and at least one of regional amplitude or regional phase of the oscillation in at least one region of interest to determine a first impedance quantification 401. The frequency of oscillation may then be changed in a first maneuver 403, for example, decreased to about 5 Hz. With the patient oscillating at the new frequency, the EIT system may again measure the frequency and at least one of regional amplitude or phase to determine a second impedance quantification 402.

Based on the first impedance quantification 401, the second impedance quantification 402, and the change in oscillation frequency associated with the first maneuver 403, the EIT system may calculate at least one first ventilatory parameter 408, including regional lung compliance, regional lung resistance, or a combination thereof. The at least one parameter associated with the maneuver may be received from a mechanical ventilator, a pressure sensor, a flow sensor, a user input, or combinations thereof. In some embodiments, the at least one parameter associated with the maneuver is instead calculated from the impedance quantifications before and after the maneuver, for example, by determining pre-maneuver and post-maneuver frequency or inspiratory-to-expiratory ratio by analyzing the corresponding impedance data. In some embodiments, the disclosed approach does not rely on data measured at the patient's airway or on data received from a ventilator. Instead, one or more parameters associated with a ventilatory maneuver may be determined from electrical impedance data acquired before and after the maneuver.

In some embodiments, the MAP is then changed in a second maneuver 404, for example, decreased to about 6 cmH₂O. After the second maneuver 404, the EIT system may measure frequency and regional amplitude to establish a third impedance quantification 405. A third maneuver 407 may then change oscillation frequency again, for example, back to about 8 Hz. After the third maneuver 407, the EIT system may measure frequency and at least one of regional amplitude or phase to establish a fourth impedance quantification 406. The EIT system may then calculate at least one second ventilatory parameter 409 based on the third impedance quantification 405, the fourth impedance quantification 406, and changes in oscillation frequency associated with the third maneuver 407. In some embodiments, the first ventilatory parameter 408 and the second ventilatory parameter 409 are compared to determine whether regional lung compliance, regional lung resistance, or both increase or decrease after the second maneuver 404, thereby providing a ventilatory figure of merit.

In some embodiments, once regional lung characteristics have been obtained, the system calculates and displays how at least one of a spontaneous component, a conventional-ventilation component, or an HFV component of ventilation would behave in terms of at least one of distribution of volume, regional distribution amplitude of tidal volume or oscillation, regional delays, time constants, or dynamics of lung inflation if a selected mechanical ventilation maneuver were performed.

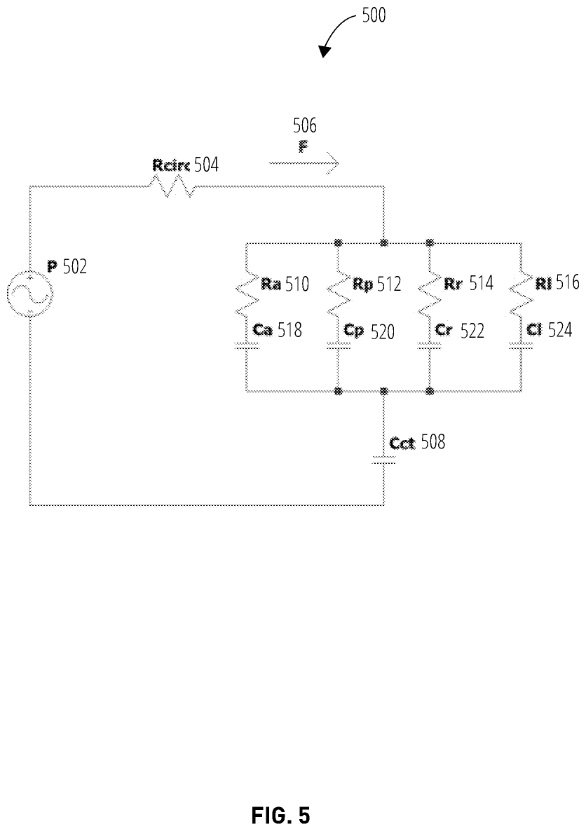

FIG. 5 illustrates a lumped-parameter circuit schematic 500 representative of a respiratory system that may be used to determine regional lung compliance, regional lung resistance, or both. In the illustrated embodiment, a pressure source P 502 may represent pressure applied by a ventilator, and Rcirc 504 may represent resistance associated with a ventilator circuit. The lung may be represented by a plurality of compartment branches arranged in parallel, each branch including a respective resistance term and a respective compliance term. In the illustrated example, the compartment branches may correspond to selected regions of interest. By way of example, the selected regions of interest may include an anterior region, a posterior region, a right region, and a left region.

In the illustrated model, F 506 may denote airflow; V (not shown) may denote global volume of air in the lung system; Zeq (not shown) may denote an equivalent impedance of the parallel compartment branches shown in FIG. 5; Zct (not shown) may denote an impedance associated with the chest-wall compliance term Cct 508; Cct 508 may denote chest-wall compliance; Va, Vp, Vr, and VI (not shown) may denote volume of air associated with the selected regions of interest; j may denote the imaginary unit; ω may denote angular frequency; f (not shown) may denote fundamental ventilation frequency; Ra 510, Rp 512, Rr 514, and Rl 516 may denote compartmental airflow resistances; and Ca 518, Cp 520, Cr 522, and Cl 524 may denote compartmental compliances.

In one illustrated implementation, the relationships of the model may be expressed as:

$$F = \frac{P}{{R}_{circ} + {Z}_{eq} + {Z}_{ct}} \qquad ( 3 )$$

$${Z}_{eq} = {( \frac{1}{{Z}_{a}} + \frac{1}{{Z}_{p}} + \frac{1}{{Z}_{r}} + \frac{1}{{Z}_{l}} )}^{- 1} \qquad ( 4 )$$

$${Z}_{a} = {R}_{a} + \frac{1}{{j ω C}_{a}} \qquad ( 5 )$$

$${Z}_{p} = {R}_{p} + \frac{1}{{j ω C}_{p}} \qquad ( 6 )$$

$${Z}_{r} = {R}_{r} + \frac{1}{{j ω C}_{r}} \qquad ( 7 )$$

$${Z}_{l} = {R}_{l} + \frac{1}{{j ω C}_{l}} \qquad ( 8 )$$

$${Z}_{ct} = \frac{1}{{j ω C}_{ct}} \qquad ( 9 )$$

$$ω = 2 π f \qquad ( 10 )$$

$${V}_{a} = \frac{1}{j ω} \cdot \frac{1}{{R}_{a} + \frac{1}{{j ω C}_{a}}} \cdot \frac{{Z}_{eq}}{{R}_{circ} + {Z}_{eq} + {Z}_{ct}} \cdot P \qquad ( 11 )$$

$${V}_{p} = \frac{1}{j ω} \cdot \frac{1}{{R}_{p} + \frac{1}{{j ω C}_{p}}} \cdot \frac{{Z}_{eq}}{{R}_{circ} + {Z}_{eq} + {Z}_{ct}} \cdot P \qquad ( 12 )$$

$${V}_{r} = \frac{1}{j ω} \cdot \frac{1}{{R}_{r} + \frac{1}{{j ω C}_{r}}} \cdot \frac{{Z}_{eq}}{{R}_{circ} + {Z}_{eq} + {Z}_{ct}} \cdot P \qquad ( 13 )$$

$${V}_{l} = \frac{1}{j ω} \cdot \frac{1}{{R}_{l} + \frac{1}{{j ω C}_{l}}} \cdot \frac{{Z}_{eq}}{{R}_{circ} + {Z}_{eq} + {Z}_{ct}} \cdot P \qquad ( 14 )$$

$$V = {V}_{a} + {V}_{p} + {V}_{r} + {V}_{l} = \frac{1}{j ω} \cdot \frac{{Z}_{eq}}{{R}_{circ} + {Z}_{eq} + {Z}_{ct}} \qquad ( 15 )$$

In some embodiments, Eqs. (3)-(15) are applied on a compartment-by-compartment basis, on a region-of-interest basis, on a pixelwise basis, or on a global basis. For multiple ventilation fundamental frequencies, the system may obtain global delivered volume V, compartmental volumes Va, Vp, Vr, and Vl, or pixelwise volume values. The maneuver may include varying oscillation frequency f across a range of values. For each frequency, corresponding global or regional volume values may be measured, and data sets such as (fi, Vi, Vai, Vpi, Vri, Vli) may be recorded. A curve-fitting procedure may then be performed using the theoretical relationships of Eqs. (3)-(15). The fitting procedure may estimate Ra 510, Rp 512, Rr 514, Rl 516, Ca 518, Cp 520, Cr 522, and Cl 524, pixelwise compliance and resistance, or global compliance and resistance, by minimizing a selected error function between measured values and values calculated by the model. In some embodiments, the fitting is performed by least squares, another optimization algorithm, or another numerical minimization procedure.

In some embodiments, the system identifies one or more regions that are silent or under-responsive to HFV, spontaneous breathing, or conventional ventilation, and may use regional compliance, regional resistance, oscillation magnitude, oscillation phase, baseline behavior, or combinations thereof to select or adjust one or more HFV parameters, including frequency, mean airway pressure (MAP), oscillatory amplitude, inspiratory time, inspiratory-to-expiratory ratio, or combinations thereof, to improve homogeneity of oscillation distribution, recruit one or more regions, or improve distribution of tidal volume.

In some embodiments, the identified regions include HFV silent regions, conventional silent regions, full silent regions, or combinations thereof. An HFV silent region may include a region in which conventional ventilation or spontaneous breathing is observed while little or no HFV oscillation is observed. A conventional silent region may include a region in which HFV oscillation is observed while little or no conventional ventilation or spontaneous breathing is observed. A full silent region may include a region in which lung tissue is expected, for example, based on population-based anatomy, pulsatility, or both, while neither HFV oscillation nor tidal-volume variation associated with conventional ventilation or spontaneous breathing is observed.

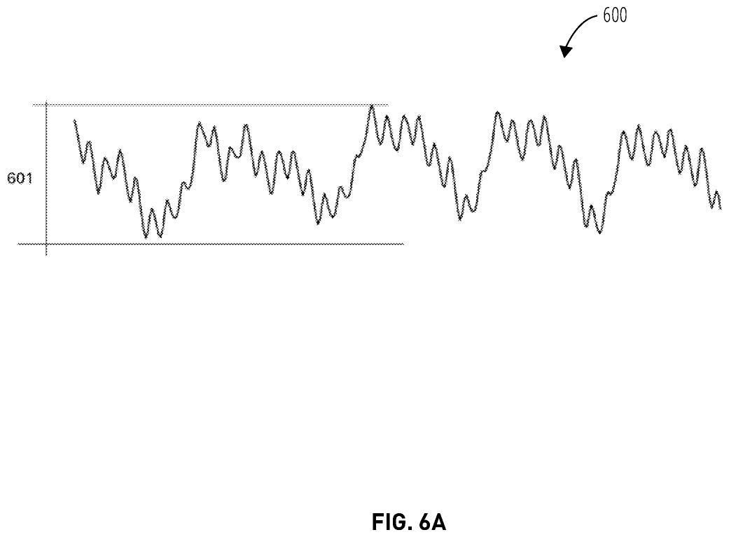

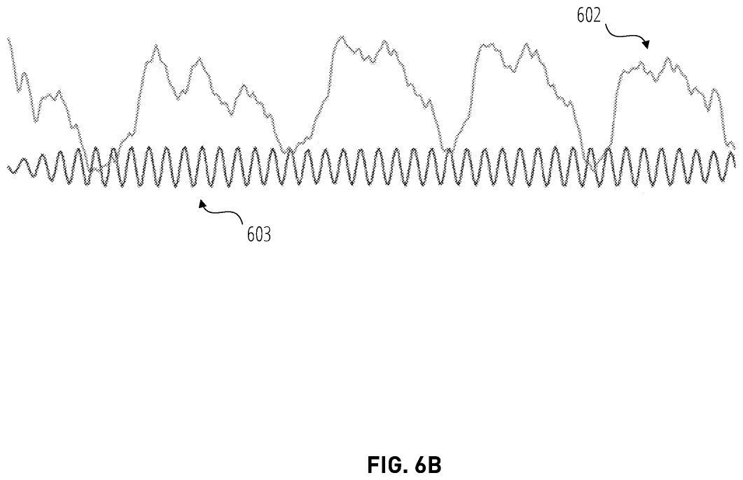

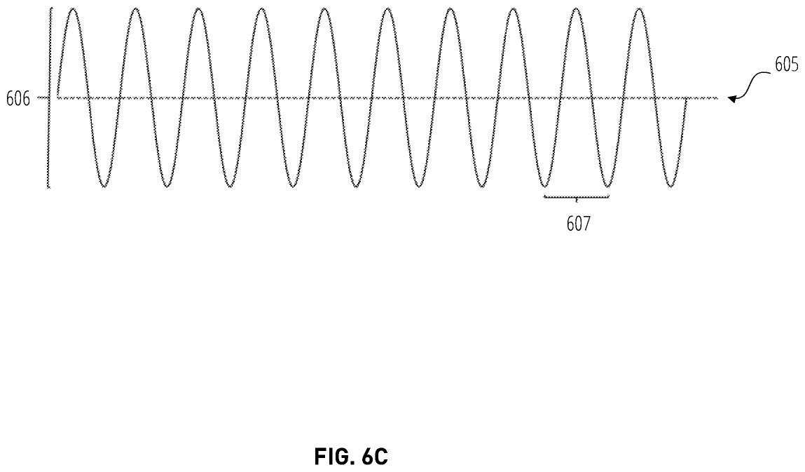

With reference to FIGS. 6A-6C, a plethysmogram 600 may represent a sum of regional impedance variation over time. FIG. 6A may represent the plethysmogram 600 including an HFV component 603 and a spontaneous-breathing component 602. The amplitude 601 of the plethysmogram 600 may correspond to a combined amplitude associated with the HFV component 603 and the spontaneous-breathing component 602. FIG. 6B may illustrate separation of the spontaneous-breathing component 602 and the HFV component 603. In some embodiments, a cardiogenic oscillation component is still present after such separation and may be filtered later. FIG. 6C may illustrate the HFV component 603 after separation. In the illustrated example of FIG. 6C, a mean airway pressure 605, an airway pressure amplitude 606, and an oscillation frequency 607 may be schematically indicated.

By calculating a power spectrum of the plethysmogram 600, the algorithm may detect the oscillation frequency 607 associated with the HFV component 603, in some embodiments assuming a range between about 5 Hz and about 20 Hz. A band-pass filter with low and high cutoff frequencies about 1 Hz away from the oscillation frequency 607 may then be used to obtain the HFV component 603. The resulting HFV component 603 may then be subtracted from a raw signal represented by the plethysmogram 600 to obtain the spontaneous-breathing component 602, when present. For a given signal, the power spectrum may provide a plot of the portion of the signal's power falling within selected frequency bins. A power spectral density of the signal may describe power present in the signal as a function of frequency, per unit frequency, for example, in W/Hz. The power spectrum may be generated using a discrete Fourier transform or another spectral-estimation technique, including a maximum entropy method.

The regional assessment of signal magnitude may serve as a proxy for regional distribution of tidal volume delivered to the patient. The regional assessment of signal baseline may serve as a proxy for regional distribution of aeration or functional residual capacity. The regional assessment of signal delay may serve as a proxy for regional distribution of a resistance-compliance time constant. In some embodiments, the system acquires impedance signals over several oscillation cycles and may generate a representative oversampled cycle to show regional dynamics of inflation and to identify regions exhibiting delayed inflation.

One or more additional plethysmogram-derived quantities may be determined for a region of interest, including a plethysmogram offset, a plethysmogram average, or both. The plethysmogram offset may represent a displacement of the oscillatory impedance signal relative to a selected reference level, and the plethysmogram average may represent an average value of the oscillatory impedance signal over one or more oscillation cycles, one or more breathing cycles, or another selected time window. The plethysmogram offset, the plethysmogram average, or both may be used, for example, as indicators associated with regional aeration, regional baseline impedance behavior, regional ventilatory state, or changes in regional lung behavior before and after a ventilatory maneuver.

Impedance data may be acquired at a rate of from about 30 frames per second to about 200 frames per second, and acquisition may extend over at least one oscillation cycle. Because the impedance signals associated with the plethysmogram 600 and the HFV component 603 may be periodic and bandlimited, interpolation, fitting, demodulation, or combinations thereof may be used to obtain a higher effective sampling rate within the bandwidth of the acquired signal. In some embodiments, these techniques facilitate estimation of oscillation amplitude, oscillation phase, delayed inflation, or combinations thereof.

In some embodiments, a drop in baseline impedance behavior or aeration in a given region suggests that one or more HFV parameters, including the mean airway pressure 605, the airway pressure amplitude 606, the oscillation frequency 607, or combinations thereof, are not sufficient to keep that region inflated. In some embodiments, regions that inflate or deflate earlier or later than other regions are subjected to greater mechanical stress than more synchronous regions. Accordingly, assessment of regional delay, delayed inflation, delayed deflation, or combinations thereof may be used to select, recommend, or adjust one or more HFV parameters, including the mean airway pressure 605, the airway pressure amplitude 606, the oscillation frequency 607, or combinations thereof, to improve synchrony of regional lung behavior.

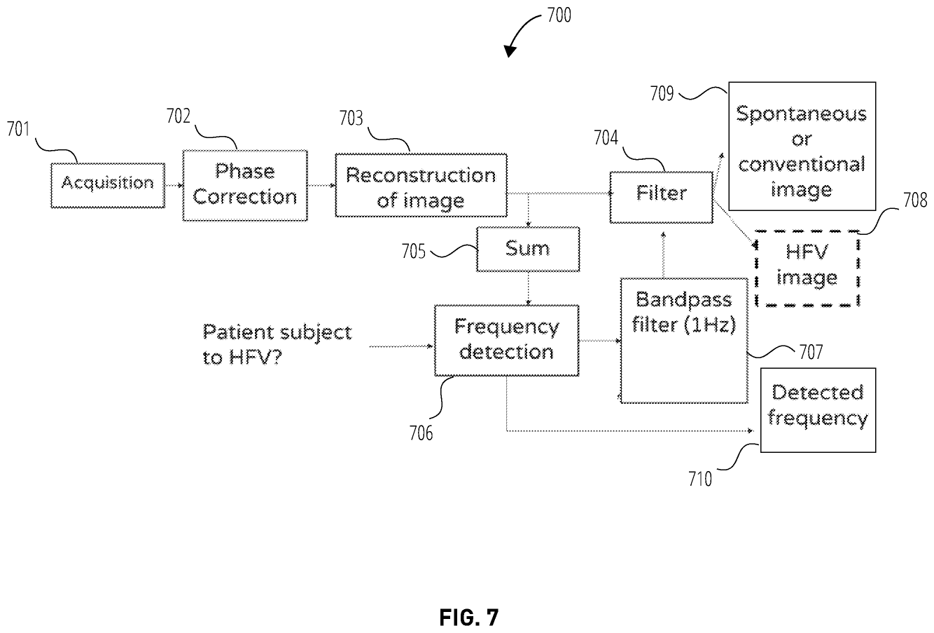

FIG. 7 illustrates a data-flow diagram of a process 700 used to obtain regional impedance data representing HFV ventilation. An act 701 may include acquisition of electrical impedance data. An act 702 may include phase correction. A data frame may represent a complete set of electrical-potential measurements acquired from all electrode pairs during one full cycle of sequential current injections. Because the current source may be sequentially connected to each electrode pair within a data frame, a natural time delay may occur between acquired electrical-potential data points. This delay may be negligible for lower-frequency signals, but for signals acquired during HFV the delay may become closer to the period of the signal and may distort the EIT images. To compensate, fractional sample delay algorithms may be applied to resample the measured data. Such algorithms may include phase shift in the frequency domain, finite impulse response filtering, interpolation, or combinations thereof. A reconstruction-of-image act 703 may receive phase-corrected data and may generate an impedance image for further processing.

A filter act 704 and a sum act 705 may cooperate with a frequency-detection act associated with act 706 to separate image components and to determine an HFV frequency. Act 706 may include frequency detection. In some embodiments, signal amplitude is also considered to determine whether a detected HFV signal is relevant or merely noise. For example, a criterion based on absolute amplitude or on percent amplitude in a low bandwidth, for example, relative to spontaneous-breathing amplitude, may be used. HFV frequency may be detected using one or more of the following strategies: FFT-based detection; a maximum frequency error of about 0.25 Hz using an FFT with about 256 or about 512 signal samples together with zero padding; and peak-detection logic in which a peak above about 4 Hz is considered HFV only if that peak is greater than peaks between about 2 Hz and about 4 Hz so as to reduce influence of spontaneous breathing and pulsatility. In some embodiments, a detected-frequency output 710 is generated by the frequency-detection act 706.

In some embodiments, zero padding is used to increase FFT resolution. For example, if the signal has 512 samples, the signal may be completed with 7×512=3584 zeros so that the FFT resolution is improved for peak detection. In some embodiments, an output of the filter act 704 includes a spontaneous or conventional image 709.

An act 707 may include band-pass filtering, which may be performed by a band-pass-filter act associated with numeral 707. In some embodiments, the band-pass filter is a floating-point finite impulse response (FIR) filter. The filter may be about +1 Hz wide around the detected HFV frequency. In some embodiments, the filter has about 0 dB attenuation at the HFV frequency and more than about 40 dB rejection outside the passband. The filter order may be about 128 or about 256. In some embodiments, a filter order of 256 is used because lower orders may attenuate the sine wave where the actual frequency differs materially from the frequency detected by FFT. In some embodiments, unit tests are performed for multiple center frequencies, including higher frequencies near about 24 Hz and about 25 Hz, to test performance near an operating limit. In some embodiments, an output of the band-pass-filtering act 707 includes an HFV image 708.

In one example, where frequency denotes a detected center frequency in hertz (Hz), samplerate denotes a sampling rate of the acquired signal in hertz (Hz), Order denotes a filter order, Ncoeff denotes a number of filter coefficients, and b denotes a vector of FIR filter coefficients, the filter design may be expressed as Order=256, Ncoeff=Order+1, and b=fir2 (Ncoeff, [0, frequency−2, frequency−1, frequency+1, frequency+2, samplerate/2]/(samplerate/2), [0, 0, 1, 1, 0, 0]). In this example, the frequency breakpoints 0, frequency−2, frequency−1, frequency+1, frequency+2, and samplerate/2 may be expressed in hertz and normalized by the Nyquist frequency samplerate/2 for input to the fir2 design routine.

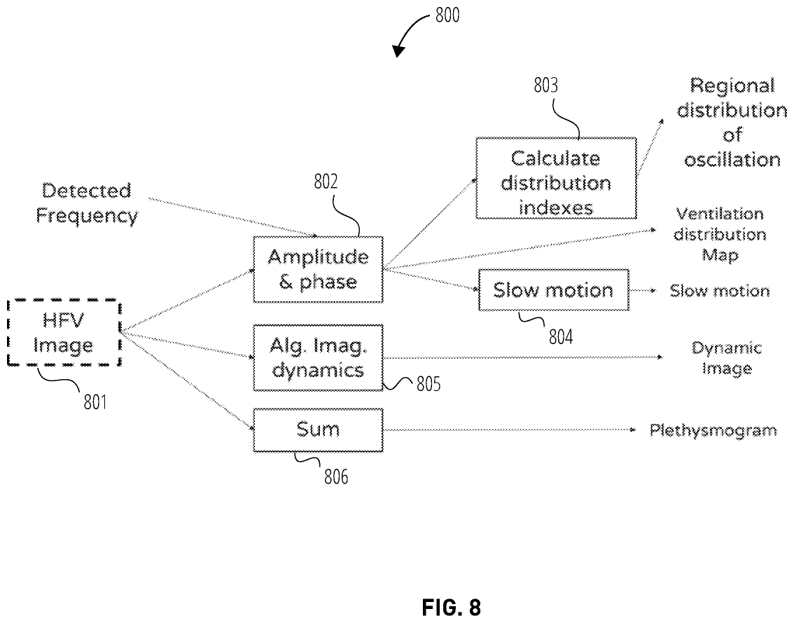

With reference to FIG. 8, a data-flow diagram 800 of impedance quantification acts performed in an embodiment is illustrated. An HFV image 801 may be provided to an amplitude-and-phase act 802, an algorithmic-image-dynamics act 805, and a sum act 806. In some embodiments, the HFV image 801 corresponds to the HFV image 708 output from the band-pass-filtering act 707 of FIG. 7. In some embodiments, the HFV image 801 is the HFV image 708 itself or may be derived therefrom.

An act associated with the amplitude-and-phase act 802 may include calculating regional distribution of oscillation after spontaneous and cardiogenic components have been filtered from the impedance data. This act may additionally allow determination of amplitude ratios, including spontaneous/HFV and cardiogenic-oscillation/HFV ratios. In some embodiments, amplitude is estimated using demodulation, which may also allow estimation of phase. The phase estimate may be used to generate a slow-motion display for any HFV frequency.

In some embodiments, alternative methods are used to estimate amplitude. For example, a max (window)-min (window) approach may be used, although that approach may have limitations at subharmonics of the sampling frequency because, depending on signal phase, sampled points may not coincide with maxima and minima of the continuous-time signal. As another example, Parseval's theorem may be used. For example, for a sampled sinusoidal signal, x[n] may denote a sampled impedance signal at sample index n, A may denote signal amplitude, f may denote oscillation frequency, fₛ may denote sampling frequency, φ may denote phase, E may denote signal energy over N samples, and θ may denote an angular argument used in the trigonometric identity below:

$$x [ n ] = A sin ( 2 π f \frac{n}{{f}_{s}} + ϕ ) \qquad ( 16 )$$

$${sin}^{2} ( θ ) = \frac{1 - cos ( 2 θ )}{2} \qquad ( 18 )$$

$${x [ n ]}^{2} = {A}^{2} {sin}^{2} ( 2 π f \frac{n}{{f}_{s}} + ϕ ) \qquad ( 19 )$$

$${x [ n ]}^{2} = \frac{{A}^{2}}{2} - \frac{{A}^{2}}{2} cos ( 4 {π f}_{0} \frac{n}{{f}_{s}} + 2 ϕ ) \qquad ( 20 )$$

$$E = {Σ x [ n ]}^{2} \approx \frac{{A}^{2}}{2} N \qquad ( 21 )$$

$$A = \sqrt{\frac{2 E}{N}} = \sqrt{\frac{2}{N} {Σ x [ n ]}^{2}} \qquad ( 22 )$$

In some embodiments, FFT-derived amplitude values are additionally or alternatively used. The amplitude-and-phase act 802 may provide amplitude and phase information to a calculate-distribution-indexes act 803, a slow-motion act 804, and the algorithmic-image-dynamics act 805. The calculate-distribution-indexes act 803 may generate a regional distribution of oscillation and a ventilation distribution map. The slow-motion act 804 may generate a slow-motion display. The algorithmic-image-dynamics act 805 may generate a dynamic image. The sum act 806 may generate a plethysmogram.

To generate the slow-motion display, synchronous demodulation may be used. Using the frequency estimated by a frequency estimator, amplitude and phase may be estimated on a pixel-by-pixel basis. This approach may be computationally simpler than FFT-based refinement using extensive zero padding. In one implementation, synchronous demodulation may include the following acts. First, frequency may be detected using FFT with padding on the plethysmograph. Second, a sine wave and a cosine wave may be generated at the detected frequency, each with amplitude 1 and phase 0, using the EIT sampling rate, for example, about 50.08 Hz. Third, projection calculations may be applied to the two generated reference signals, pixel by pixel or region by region. The result may be numerically equivalent to FFT-based analysis while providing both amplitude and phase for each pixel. Fourth, a slow-motion image may be generated by synthesizing signals for each pixel using a higher sampling frequency, for example, about 500 Hz, which may correspond to about 25 samples per cycle in the case of about 20 Hz HFV.

In some embodiments, other algorithms are used instead of or in combination with synchronous demodulation, including sample re-ordering or cycle population, and FFT on a pixel-by-pixel basis to determine both amplitude and phase. Once amplitude and phase are known pixel by pixel, the signal may be synthesized with a higher sampling rate to generate a slow-motion effect. In some embodiments, the slow-motion image is displayed at a playback speed lower than an acquisition speed of the electrical impedance data.

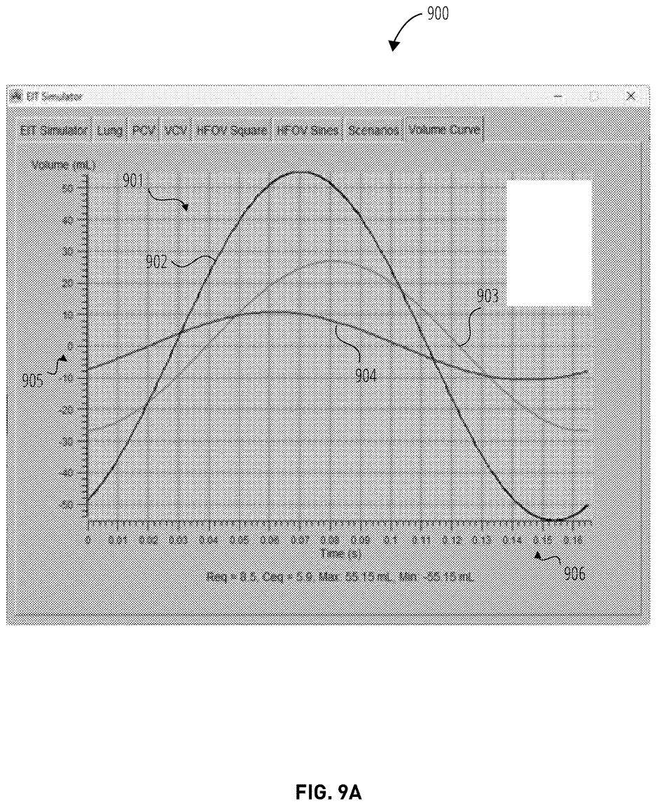

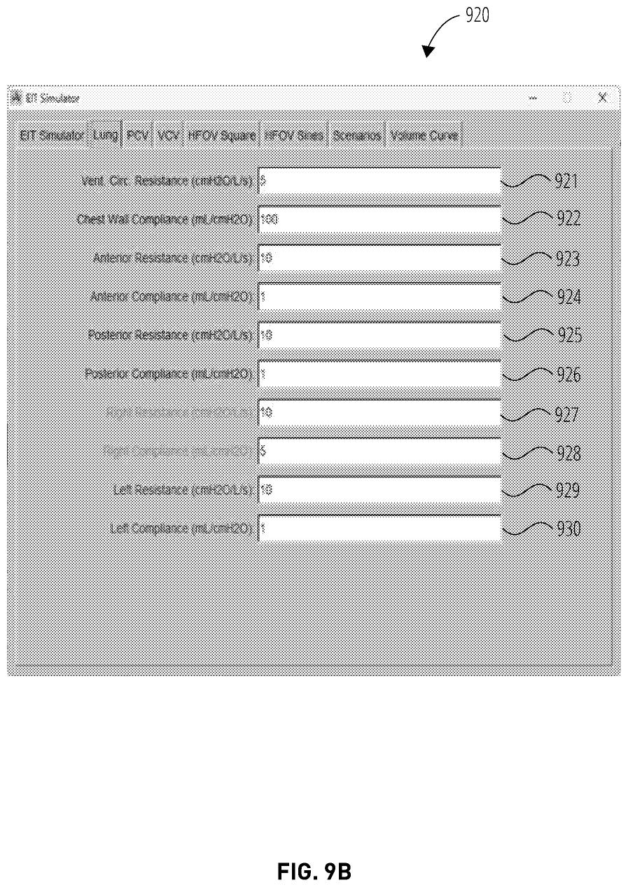

With reference to FIG. 9A, a graphical user interface 900 may include a plot 901 configured to show how an HFV component of ventilation behaves in terms of distribution of volume and phase for a selected oscillatory frequency. In the illustrated example, the plot 901 may include a global response curve 902, a right response curve 903, and a left response curve 904. The plot 901 may further include a volume axis 905, and a time axis 906.

In one example, with a frequency of about 6 Hz, the left response curve 904 may fill before and with a lower amplitude than the right response curve 903, and a selected fraction of the oscillation, for example, about 63.3%, may be directed to the right region of interest. In some embodiments, simulation of regional response employs a sinusoidal lung model of the form recited in Eq. (16), where EIT pixels correlate with regional volume curves, and where pressure (t) may be mapped through a linear model defined by resistance and compliance to generate a corresponding volume curve. In some embodiments, the amplitude term A is estimated according to Eq. (22), and the phase term φ may be estimated by synchronous demodulation. The resulting regional amplitude and phase values may then be used to simulate regional distribution of volume, regional phase delay, delayed filling, or other predicted responses to a selected ventilatory maneuver.

In some embodiments, the system uses one or more determined regional lung characteristics, one or more identified silent regions, or both, to select, recommend, or adjust one or more HFV parameters. For example, the system may use regional compliance, regional resistance, oscillation magnitude, oscillation phase, baseline impedance behavior, inflation dynamics, or combinations thereof to select, recommend, or adjust frequency, mean airway pressure, oscillatory amplitude, inspiratory time, inspiratory-to-expiratory ratio, or combinations thereof. In some embodiments, the selected or adjusted HFV parameters improve homogeneity of oscillation distribution, recruit one or more regions that are silent or under-responsive to HFV, spontaneous breathing, or conventional ventilation, improve distribution of combined tidal volume, or combinations thereof.

In some embodiments, the impedance-based system determines a homogeneity metric for one or more regions of interest, for a plurality of regions of interest, or for the lung as a whole. By way of example only, the homogeneity metric may be based on regional distribution of oscillation magnitude, regional distribution of tidal-volume-related impedance change, regional phase distribution, regional delay, regional compliance, regional resistance, or combinations thereof. In some embodiments, the homogeneity metric is referred to as homogeneity of regional ventilation, homogeneity of regional oscillation distribution, or regional lung homogeneity.

With reference to FIG. 9B, a graphical user interface 920 may allow input of lung-characteristic values used to simulate a predicted distribution of volume and phase for a selected oscillatory frequency. In the illustrated example, the graphical user interface 920 may include a ventilator-circuit-resistance input field 921, a chest-wall-compliance input field 922, an anterior-resistance input field 923, an anterior-compliance input field 924, a posterior-resistance input field 925, a posterior-compliance input field 926, a right-resistance input field 927, a right-compliance input field 928, a left-resistance input field 929, and a left-compliance input field 930. In some embodiments, values entered into the input fields 921-930 correspond to one or more model parameters used to simulate regional response, including ventilator-circuit resistance, chest-wall compliance, regional resistance, regional compliance, or combinations thereof.

In some embodiments, the system uses compliance and resistance associated with a region of interest in consideration of a difference between a first impedance quantification and a second impedance quantification, and at least one parameter associated with a maneuver. In some embodiments, one or more of the values shown in the graphical user interface 920 are entered by a user, determined from impedance data, determined from one or more prior assessments, received from another device, or combinations thereof. The entered or determined values may then be used to simulate predicted regional distribution of volume, regional phase delay, delayed filling, delayed deflation, or other predicted responses to a selected ventilatory maneuver or oscillatory frequency.

The predicted regional lung response may include one or more predicted outputs associated with the at least one region of interest, including lung homogeneity, a regional distribution of magnitude of oscillation, lung recruitment, regional asynchronous filling, a dynamic of lung inflation, a dynamic of lung deflation, a regional delay, a regional phase delay, a regional distribution of tidal volume, or combinations thereof. The predicted regional lung response may be generated from one or more determined regional lung characteristics, one or more ventilatory figures of merit, one or more identified silent or under-responsive regions, one or more user-entered model parameters, or combinations thereof.

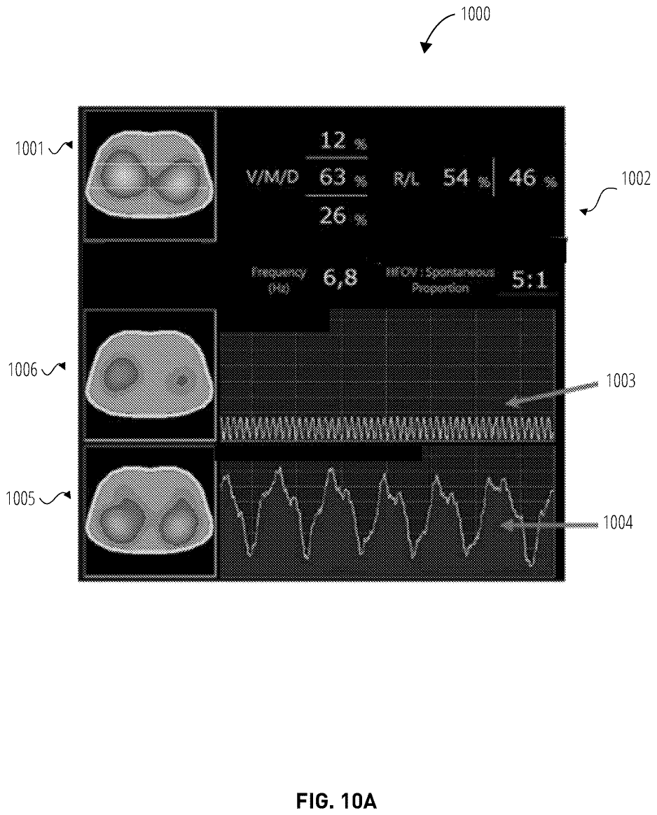

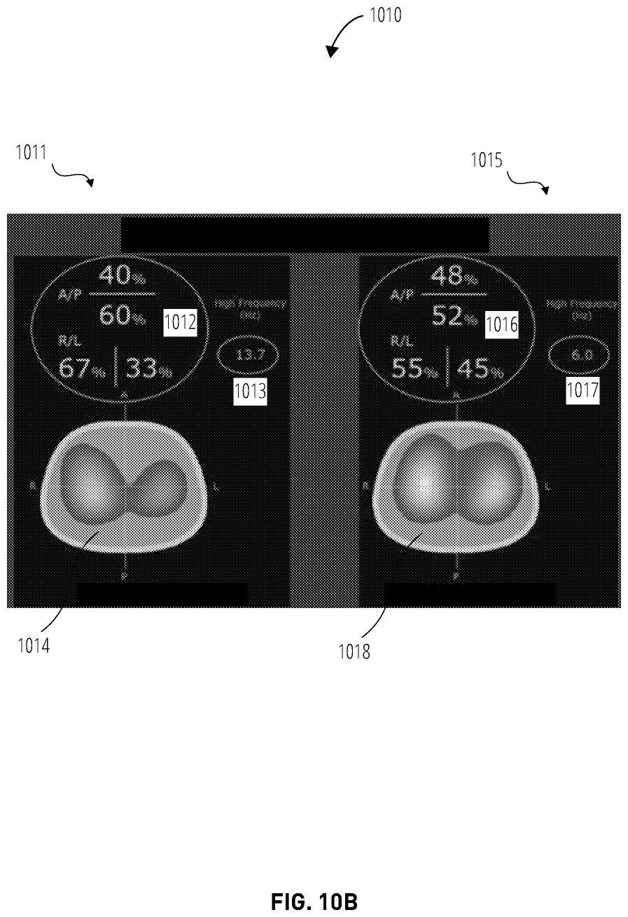

With reference to FIG. 10A, a display 1000 of an EIT device may present user-interface elements associated with ventilatory assessment. In the illustrated example, the display 1000 may include an HFV map 1001, a distribution-indices region 1002, an HFV plethysmogram 1003, a spontaneous-ventilation plethysmogram 1004, a distribution map 1005, and a slow-motion dynamic image 1006. In some embodiments, the HFV map 1001 represents regional amplitude of oscillations, and the distribution map 1005 may represent regional distribution of combined tidal volume resulting from HFV combined with spontaneous breaths. In some embodiments, the distribution-indices region 1002 includes one or more numerical indicators associated with ventilation distribution, including anterior/posterior distribution, right/left distribution, oscillation frequency, HFV-to-spontaneous proportion, or combinations thereof. In some embodiments, the slow-motion dynamic image 1006 shows regional inflation and deflation behavior over time. In some embodiments, the display 1000 additionally provides regional lung characteristics, simulated regional responses to selected maneuvers, one or more alerts, one or more recommendations, or combinations thereof.

In some embodiments, one or more regions silent to oscillation, one or more regions silent to spontaneous breaths, one or more regions silent to spontaneous breathing but not oscillation, one or more regions silent to oscillation but not spontaneous breathing, or combinations thereof are shown in different colors, textures, patterns, or other distinguishable visual treatments.

With reference to FIG. 10B, a comparative display 1010 may show changes in regional distribution associated with different oscillatory frequencies. In the illustrated example, the comparative display 1010 may include a first panel 1011 and a second panel 1015. The first panel 1011 may include a first distribution-indices region 1012, a first frequency indicator 1013, and a first distribution map 1014. The second panel 1015 may include a second distribution-indices region 1016, a second frequency indicator 1017, and a second distribution map 1018. In some embodiments, the first frequency indicator 1013 and the second frequency indicator 1017 correspond to different selected HFV frequencies, and the corresponding distribution maps 1014 and 1018 may illustrate how regional oscillation distribution, regional tidal-volume distribution, or both, change as a function of frequency. The first panel and the second panel may correspond to different candidate ventilator settings, including different oscillation frequencies, mean airway pressures, oscillatory amplitudes, inspiratory times, inspiratory-to-expiratory relationships, or combinations thereof.

In some embodiments, the comparative display 1010 is used to identify a frequency associated with a more homogeneous distribution of oscillation, improved recruitment of one or more regions, reduced silent regions, or combinations thereof. In some embodiments, one or more slow-motion dynamic images showing inflation and deflation are also presented in association with the comparative display 1010. Accordingly, embodiments of the present disclosure may provide methods, devices, systems, and computer-readable media that facilitate better configuration of HFV parameters, including frequency, MAP, oscillatory amplitude, inspiratory time, inspiratory-to-expiratory ratio, or combinations thereof, by assessing regional lung characteristics using EIT. By using regional impedance quantifications before and after controlled maneuvers, the disclosed techniques may facilitate more accurate assessment of regional lung mechanics than techniques based solely on airway measurements and may facilitate targeted ventilation strategies.

The methods and systems according to embodiments of the disclosure offer several benefits and advantages compared to conventional methods and systems. For example, the disclosed approaches may facilitate non-invasive, bedside, and region-specific assessment of lung behavior during high-frequency ventilation, rather than relying exclusively on global airway-based measurements. By using electrical impedance data acquired before and after one or more ventilatory maneuvers, the disclosed approaches may facilitate assessment of regional lung compliance, regional lung resistance, oscillation distribution, regional timing, aeration-related behavior, and other regional lung characteristics that may be difficult to evaluate using conventional techniques. In some embodiments, the disclosed approaches further facilitate identification of silent or under-responsive regions, assessment of delayed inflation or deflation, simulation of predicted regional responses to candidate ventilator-setting changes, and generation of slow-motion displays that improve visualization of regional lung dynamics. Accordingly, the disclosed methods and systems may facilitate more informed selection or adjustment of high-frequency ventilation parameters, improved homogeneity of regional ventilation, improved recruitment of one or more lung regions, and more targeted ventilatory strategies.

While embodiments of the disclosure may be susceptible to various modifications and alternative forms, specific embodiments have been shown by way of example in the drawings and have been described in detail herein. However, it should be understood that the disclosure is not limited to the particular forms disclosed. Rather, the disclosure encompasses all modifications, variations, combinations, and alternatives falling within the scope of the disclosure as defined by the following appended claims and their legal equivalents.

What is claimed is:

1. A method for assessing regional lung characteristics of a patient undergoing high-frequency ventilation using an impedance-based device, the method comprising:
determining a first impedance quantification from first electrical impedance data associated with at least one region of interest and acquired prior to a first ventilatory maneuver;
determining a second impedance quantification from second electrical impedance data associated with the at least one region of interest and acquired following the first ventilatory maneuver;
obtaining at least one parameter associated with the first ventilatory maneuver;
determining a first regional lung characteristic associated with the at least one region of interest based at least in part on the first impedance quantification, the second impedance quantification, and the at least one parameter associated with the first ventilatory maneuver; and
causing a change in a configuration of a high-frequency mechanical ventilator based at least in part on the first regional lung characteristic, wherein the change in the configuration comprises changing at least one of a frequency of oscillation, an inspiratory time, an inspiratory-to-expiratory relationship, an amplitude of oscillation, a mean airway pressure, or any combination thereof.

2. The method of claim 1, further comprising:
determining a third impedance quantification from third electrical impedance data associated with the at least one region of interest and acquired after a second ventilatory maneuver and prior to a third ventilatory maneuver;
determining a fourth impedance quantification from fourth electrical impedance data associated with the at least one region of interest and acquired following the third ventilatory maneuver;
obtaining at least one parameter associated with the third ventilatory maneuver;
determining a second regional lung characteristic associated with the at least one region of interest based at least in part on the third impedance quantification, the fourth impedance quantification, and the at least one parameter associated with the third ventilatory maneuver;
obtaining at least one parameter associated with the second ventilatory maneuver;
determining at least one ventilatory figure of merit associated with the at least one region of interest based at least in part on the first regional lung characteristic, the second regional lung characteristic, and the at least one parameter associated with the second ventilatory maneuver; and
outputting at least one of the third impedance quantification, the fourth impedance quantification, the second regional lung characteristic, or the at least one ventilatory figure of merit associated with the at least one region of interest.

3. The method of claim 1, wherein determining the first regional lung characteristic comprises determining one or more of regional lung compliance, regional lung resistance, or lung homogeneity.

4. The method of claim 1, wherein determining the first impedance quantification comprises correcting a phase of electrical impedance data corresponding to the first impedance quantification by applying at least one of a fractional sample delay algorithm, a phase shift in a frequency domain, finite impulse response filtering, or interpolation to the corresponding electrical impedance data.

5. The method of claim 1, wherein determining the first impedance quantification comprises determining one or more of a baseline of a plethysmogram, an offset of the plethysmogram, an average of the plethysmogram, a magnitude of oscillation, a phase of oscillation, and an asynchronous filling.

6. The method of claim 5, wherein determining the first impedance quantification comprises determining a magnitude of oscillation, and wherein determining the magnitude of oscillation comprises:
obtaining a frequency of oscillation;
generating a sine wave and a cosine wave at the frequency of oscillation; and
applying a calculation of projections to the sine wave and the cosine wave on a pixel-by-pixel basis or a region-by-region basis.

7. The method of claim 1, wherein the first ventilatory maneuver comprises changing at least one parameter, the at least one parameter comprising a frequency of oscillation, an inspiratory time, an inspiratory-to-expiratory relationship, an amplitude of oscillation, or a mean airway pressure.

8. The method of claim 1, wherein obtaining the at least one parameter associated with the first ventilatory maneuver comprises at least one of receiving the at least one parameter from a mechanical ventilation device and measuring the at least one parameter with a pressure sensor or a flow sensor coupled to the impedance-based device.

9. The method of claim 1, wherein obtaining the at least one parameter associated with the first ventilatory maneuver comprises assessing the at least one parameter by analyzing electrical impedance data acquired prior to the first ventilatory maneuver and electrical impedance data acquired following the first ventilatory maneuver.

10. A system for displaying slow-motion electrical impedance data associated with at least one region of interest of a patient undergoing high-frequency ventilation, the system comprising:
an impedance-based device comprising:
a plurality of electrodes; and
a controller coupled to the plurality of electrodes, the controller comprising:
an interface module configured to collect electrical impedance data from the plurality of electrodes;
at least one processor; and
thereon that, when executed by the at least one processor, cause the controller to:
acquire electrical impedance data corresponding to a plurality of oscillations;
estimate, from the electrical impedance data, at least one of an amplitude or a phase associated with the at least one region of interest;
generate a slow-motion image associated with the at least one region of interest by synthesizing a signal based at least in part on the at least one of the amplitude or the phase; and
output the slow-motion image via at least one display of the impedance-based device.

11. The system of claim 10, wherein estimating the at least one of the amplitude or the phase comprises the instructions causing the controller to:
obtain a frequency of oscillation;
generate a sine wave and a cosine wave at the frequency of oscillation; and
apply a calculation of projections to the sine wave and the cosine wave on a pixel-by-pixel basis or a region-by-region basis.

12. The system of claim 10, wherein outputting the slow-motion image comprises the instructions causing the controller to display the slow-motion image at a playback speed lower than an acquisition speed of the electrical impedance data.

13. The system of claim 10, wherein generating the slow-motion image comprises the instructions causing the controller to synthesize signals associated with the at least one region of interest using a sampling frequency higher than a sampling frequency used to acquire the electrical impedance data.

14. The system of claim 10, wherein the instructions, when executed by the at least one processor, further cause the controller to separate a high-frequency ventilation component from at least one of a spontaneous-breathing component or a cardiogenic component of the electrical impedance data before estimating the at least one of the amplitude or the phase.

15. The system of claim 10, wherein outputting the slow-motion image comprises the instructions causing the controller to output a dynamic image showing regional inflation and deflation over time.

16. A method for simulating a regional lung response of a patient undergoing high-frequency ventilation using an impedance-based device, the method comprising:
obtaining at least one of a first regional lung characteristic, a second regional lung characteristic, or a ventilatory figure of merit associated with at least one region of interest;
calculating a predicted regional lung response of the patient to a candidate change in a configuration of a high-frequency mechanical ventilator before the candidate change is applied based at least in part on the at least one of the first regional lung characteristic, the second regional lung characteristic, and the ventilatory figure of merit, wherein the candidate change in the configuration of the high-frequency mechanical ventilator comprises a change to at least one of a frequency of oscillation, an inspiratory time, an inspiratory-to-expiratory relationship, an amplitude of oscillation, a mean airway pressure, or any combination thereof, and
outputting the predicted regional lung response associated with the at least one region of interest via a display of the impedance-based device.

17. The method of claim 16, wherein calculating the predicted regional lung response comprises calculating at least one of lung homogeneity, a regional distribution of magnitude of oscillation, lung recruitment, asynchronous filling, a dynamic of lung inflation, a dynamic of lung deflation, a regional delay, a regional phase delay, a regional distribution of tidal volume, or any combination thereof.

18. The method of claim 16, wherein calculating the predicted regional lung response comprises calculating the predicted regional lung response based at least in part on one or more user-entered model parameters.

19. The method of claim 18, wherein the one or more user-entered model parameters comprise at least one of ventilator-circuit resistance, chest-wall compliance, regional resistance, or regional compliance.

20. The method of claim 16, wherein outputting the predicted regional lung response comprises outputting a comparative display including a first panel associated with a first candidate ventilator setting and a second panel associated with a second candidate ventilator setting, wherein the first candidate ventilator setting and the second candidate ventilator setting differ in at least one of oscillation frequency, mean airway pressure, oscillatory amplitude, inspiratory time, inspiratory-to-expiratory relationship, or any combination thereof.

21. The method of claim 16, wherein calculating the predicted regional lung response is based at least in part on one or more identified silent or under-responsive regions.

22. The method of claim 16, wherein calculating the predicted regional lung response comprises calculating at least one of a regional distribution of volume, a regional phase delay, delayed filling, delayed deflation, or any combination thereof based at least in part on regional amplitude values and regional phase values associated with the at least one region of interest.

23. A system for assessing regional lung characteristics of a patient undergoing high-frequency ventilation using an impedance-based device, the system comprising:
an impedance-based device comprising:
a plurality of electrodes; and
a controller coupled to the plurality of electrodes, the controller comprising:
an interface module configured to collect electrical impedance data from the plurality of electrodes;
at least one processor; and
thereon that, when executed by the at least one processor, cause the controller to:
determine a first impedance quantification from first electrical impedance data associated with at least one region of interest and acquired prior to a first ventilatory maneuver;
determine a second impedance quantification from second electrical impedance data associated with the at least one region of interest and acquired following the first ventilatory maneuver;
obtain at least one parameter associated with the first ventilatory maneuver; and
determine a first regional lung characteristic associated with the at least one region of interest based at least in part on the first impedance quantification, the second impedance quantification, and the at least one parameter associated with the first ventilatory maneuver, the first regional lung characteristic comprising one or more of regional lung compliance, regional lung resistance, or lung homogeneity,
wherein determining the first impedance quantification comprises:
determining one or more of a baseline of a plethysmogram, an offset of the plethysmogram, an average of the plethysmogram, a magnitude of oscillation, a phase of oscillation, and an asynchronous filling; and
correcting a phase of electrical impedance data corresponding to the first impedance quantification by applying at least one of a fractional sample delay algorithm, a phase shift in a frequency domain, finite impulse response filtering, or
interpolation to the corresponding electrical impedance data.
